(19) United States
(12) Patent Application Publication
(10) Pub. No.: US 2006/0177500 A1
(43) Pub. Date: Aug. 10, 2006

(54) Solid dispersion of tacrolimus

(76) Inventors: Hee-Jong Shin, Gyeonggi-do (KR); Jong-Lae Lim, Chungcheongnam-do (KR); Min-Hyo Ki, Chungcheongnam-do (KR); Ji-Hun Yun, Chungcheongnam-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/563,972

(22) PCT Filed: Jul. 9, 2004
(86) PCT No.: PCT/KR04/01684

§ 371 Date: Jan. 6, 2006

(30) Foreign Application Priority Data
Jul. 9, 2003 (KR) 10-2003-0046550

(51) Int. Cl. A61K 9/20

(52) U.S. Cl. 424/464

(57) ABSTRACT

The present invention relates to the carrier of the solid dispersion of tacrolimus, which is prepared by using the solid surfactant having a property of HLB value higher than or equal to about 7. The surfactants carry out a function of a carrier and a function of a dissolution enhancer, simultaneously. As a result, the dissolution rate of tacrolimus is improved, and the oral absorbability and the bioavailability may be increased due to rapid drug release.

### TECHNICAL FIELD

The present invention relates to drug carrier of the solid dispersion of water-insoluble drug tacrolimus. In particular, the present invention relates to surfactants that are able to be not only a drug carrier of solid dispersion but also a dissolution enhancer. The surfactants are solid phase at room temperature, and their HLB values are higher than or equal to about 7. Oral absorbability and bioavailability of tacrolimus may be increased due to improved dissolution rate of the solid dispersion in the present invention.

### BACKGROUND ART

There have been numerous efforts to improve dissolution rate of water-insoluble drug. These include, (a) reducing drug particle size to increase surface area, (b) solubilization in surfactant, (c) forming into micro-emulsion, (d) decreasing crystallinity of drug by formation of solid dispersion, and so on. The solid dispersion is a pharmaceutical formulation of an amorphous drug was dispersed in a solid carrier. To prepare solid dispersion, it was prepared by dissolving drug and solid carrier in organic solvent or fusing them, and then drying or cooling.

The drug used in the present invention is 17-allyl-1,14-dihydroxy-12-[2-(4-hydroxy-3-methoxycyclohexyl)-1 -methylvinyl]-23,25-dimethoxy-13,19,21,27-tetramethyl-11,28-dioxy-4-azatricycol[22.3.1.0.^{4.9}]octacos-18-ene-2,3,10,16-tetraone (hereinafter, referred to as ‘tacrolimus’). The tacrolimus possesses pharmacological activities such as immunosuppressive activity and antimicrobial activity as described in the published European patent publication No. 181462 (Publication date: Jun. 11, 1986) and therefore is useful for treatment and prevention of rejection by transplantation graft-versus-host disease by medulla ossium transplantation, auto-immune disease, infectious disease, and the like.

However, when orally administered, absorbability and bioavailability of tacrolimus are low due to insolubility of the drug in water. So tacrolimus has some disadvantages in oral administration.

Japan Patent Laid-open No. so 62-277321 has disclosed a solid dispersion comprising a water-insoluble drug of tacrolimus and a drug carrier of water-soluble polymer, however it is generally acknowledged that the absorption of such a solid dispersion after oral administration has a tendency of a large variation.

In addition, U.S. Pat. No. 6,346,537 has disclosed a pharmaceutical composition comprising a water-insoluble active substance having a tacrolimus, a surfactant(s), and a pharmaceutically acceptable solid carrier is selected from the group consisting of water-soluble polymers, saccharides and light anhydrous silicic acid. The solid carrier alone does not still increase the dissolution rate of tacrolimus as same as the solid dispersion that Japan Patent Laid-open No. so 62-277321. Therefore, it was proposed that tarolimus and a surfactant(s) are simultaneously dispersed in the solid carrier. However, in this case, the surfactant was only used for solubilization of the tarolimus, and was not used for the carrier of tacrolimus.

Korean Patent Laid-open No. 2001-0006070 has disclosed a pharmaceutical composition comprising the water-insoluble drug and two or more surfactants. But, in this case, the conventional composition is disclosed as a liquid composition, in which one surfactant dissolves the water-insoluble drug and the other surfactant. Also, the surfactant is only used for the solubilization of the water-insoluble drug in solution. Thus, the conventional composition is not related to the present invention for developing the solid form to be administered orally.

And, Korean Patent Laid-open No.2003-0040556 has described a sustained-release formulation comprising a solid dispersion of a macrolide compound. And the macrolide compound is dispersed at an amorphous state in a solid carrier that is used singly or combination of the water-soluble base (ex. water-soluble polymer), water-insoluble base (ex. wax, water-insoluble polymer).

The above-mentioned Korean Patent Laid-open No.2003-0040556 has also disclosed that disintegrators (croscarmellose sodium, carboxymethyl cellulose calcium, low substituted hydroxypropyl cellulose, starch sodium starch glycolate, microcrystalline cellulose, crospovidone, etc.) or surfactants (polyoxyethylene castor oil, polyoxyl 40 stearate, polysorbate 80, sodium lauryl sulfate, sucrose fatty acid ester (HLB≧10)) may be added to the solid dispersion for increasing the initial dissolution rate of the drug. But, small quantity of the surfactant was only used for increasing the initial dissolution rate when the drug release was over-sustained. It is not used for the drug carrier of the solid dispersion.

Above-mentioned solid dispersions are disadvantageous on the bioavailability when orally administrated due to the dissolution rate of limited.

The inventors of the present invention have made efforts to solve the problems of conventional technology as described above and to develop the effective carrier of solid dispersion, which may carry out the function of the carrier and the function of the dissolution enhancer. As a result, the inventors have known that the solid surfactant having a property of the HLB value higher than or equal to about 7 is effective as the carrier of solid dispersion. As a result, the dissolution rate of tacrolimus was improved, and the bioavailability and the oral absorbability may be increased due to excellent dissolution rate. The solid dispersion was also produced easily and stably by using a spray-dryer or a fluid bed granulator.

### DISCLOSURE OF THE INVENTION

Technical Problem

The present invention provides solid dispersion of tacrolimus improved dissolution rate, and increased oral absorbability and bioavailability due to an excellent dissolution.

The present invention also provides solid dispersion carrier that carry out a function as a drug carrier and a function as a dissolution enhancer, simultaneously.

The present invention still also provides solid dispersion that is prepared by using surfactant as the drug carrier of the solid dispersion. The surfactant has properties of hydrophile lipophile balance (HLB) value higher than or equal to about 7 and solid phase at room temperature. In addition, the present invention provides a method of processing the solid dispersion and oral dosage form using the solid dispersion.

Technical Solution

To accomplish the above-mentioned object, the present invention provides solid surfactant having a property of HLB value higher than or equal to about 7 as the carrier of the solid dispersion of tacrolimus. The surfactant can carry out a function of a carrier and a function of a dissolution enhancer, simultaneously.

The present invention also provides solid dispersion of tacrolimus such that dissolution rate is improved, and oral absorbability and bioavailability may be increased due to rapid dissolution rate.

The present invention still also provides a method of processing solid dispersion of tacrolimus and oral dosage form using the solid dispersion.

Hereinafter, the present invention is described in detail.

The present invention uses solid surfactants having a property of hydrophile lipophile balance (HLB) value higher than or equal to about 7 as the drug carrier of the solid dispersion of tacrolimus.

The surfactant is one or more selected from the group consisting of sodium lauryl sulfate(HLB=40), poloxamer(poloxamer 188, poloxamer 237, poloxamer 338, poloxamer 407) having a property of the HLB value higher than or equal to about 7, sucrose fatty acid ester(sucrose stearic acid, sucrose oleic acid, sucrose palmitic acid, sucrose miristic acid, sucrose lauric acid etc.) having a property of the HLB value of about 7 to about 18. The surfactant is not limited as above-mentioned. The solid surfactant having a property of the HLB value higher than or equal to about 7 is available. The drug and the surfactant may be preferably used by weight in ratio from 1:0.1 to 1:100, more preferably from 1:3 to 1:50.

The present invention uses the solid surfactant as the drug carrier of the solid dispersion of tacrolimus. The solid dispersion is sufficient to improve the dissolution rate, and it may increase the oral absorbability and the bioavailability of tacrolimus.

The solid dispersion is prepared by dissolving and/or dispersing tacrolimus and the solid surfactant simultaneously in organic solvent, and then by vacuum-drying for removing the organic solvent, and then by pulverization. Further, the solid dispersion may be prepared by using a spray-dryer or a fluid bed granulator. In the present invention, the surfactant is dissolved or dispersed in organic solvent with tacrolimus to act as the drug carrier of the solid dispersion.

The present invention may use any pharmaceutically acceptable solvent that is one or more selected from the group of ethanol, isopropyl alcohol, dichloromethane and chloroform, etc., and not limited as the above-mentioned solvent.

The solid dispersion of tacrolimus in the present invention may be prepared by dissolving or dispersing the tacrolimus and the solid surfactant in the proper organic solvent, and by vacuum drying for removing the organic solvent, and then by spray drying of the solution or by granulating at fluid bed granulator.

In the preparation of the solid dispersion, pharmaceutically acceptable additives such as excipients (starch, etc.), disintegrators (croscarmellose sodium, carboxymethyl cellulose calcium, low substituted hydroxypropyl cellulose, sodium starch glycolate, microcrystalline cellulose, crospovidone, etc.), coloring agents, flavouring agents, sweetening agents, and lubricants (magnesium stearate, calcium stearate, talc, etc.) may be added into the solution, optionally.

In addition, not only the above-mentioned additives but also the pharmaceutically acceptable additives such as lactose, talc and anhydrous dibasic calcium phosphate may be used for granulating-seed in the fluid bed granulator. The additives used as the seed such as lactose, talc and anhydrous dibasic calcium phosphate are not necessary for preparation of the solid dispersion of tacrolimus. They are just only the seed for fluid bed granulation. That is, the additives are not used for the drug carrier of the solid dispersion.

The pharmaceutically acceptable excipients, disintegrators, binders, coloring agents, stabilizers, sweetening agents or lubricants may be added to the the solid dispersion particle of the present invention, and the mixture may be hardly pressed and milled. As a result, fluidity and content uniformity of the prepared powder are improved. So the powder is easy to formulate in capsule or tablet.

The solid dispersion of tacrolimus in the present invention has the high dissolution rate and excellent stability, as a result, the oral absorbability and the bioavailability may be improved without variation.

The solid dispersion of the present invention may be used in a pharmaceutical preparation for oral administration and also may be converted into various dosage forms such as powders, granules, capsules, tablets, and the like, according to a conventional manner. If desired, the pharmaceutically acceptable excipients, disintegrators, binders, coloring agents, stabilizers, sweetening agents, lubricants, coating agents, or plasticizers and the like may be used for preparing pharmaceutical dosage form.

Advantageous Effects

The carrier of the solid dispersion in the present invention improves the dissolution rate of water-insoluble drug tacrolimus, so the oral absorbability and the bioavailability of tacrolimus may be increased due to rapid drug release.

The surfactant used in the present invention as the drug carrier may carry out the function of a carrier and the function of a dissolution enhancer simultaneously.

Also, the pharmaceutical dosage form provided in the present invention may improve the bioavailability and the oral absorbability of tacrolimus.

### BRIEF DESCRIPTION OF THE DRAWINGS

FIG. 1 represents a comparative graph of the dissolution rate of the solid dispersions prepared in Example 26 and Comparative examples.

### BEST MODE FOR CARRYING OUT THE INVENTION

The following examples are intended to describe the present invention in further detail and should not be constructed as limiting the scope of the invention.

### COMPARATIVE EXAMPLE 1

### Preparation of the Solid dispersion of Tacrolimus with the Surfactant its HLB Value is Low

Tacrolimus(1 g) was dissolved in the mixture of ethanol(10 ml) and dichloromethane(5 ml). To thus obtained solution, the sucrose fatty acid ester(HLB=7, 3 g) was dispersed as the drug carrier. The solution was evaporated under reduced pressure using a vacuum dryer. After drying, the residual product was pulverized.

### COMPARATIVE EXAMPLE 2

### Preparation of the Solid Dispersion of Tacrolimus with the Surfactant its HLB Value is Low

Tacrolimus(1 g) was dissolved in the mixture of ethanol(10 ml) and dichloromethane (5 ml). To thus obtained solution, the sucrose fatty acid ester(HLB=6, 3 g) was dispersed as the drug carrier. The solution was evaporated under reduced pressure using a vacuum dryer. After drying, the residual product was pulverized.

### COMPARATIVE EXAMPLE 3

The prograf 1 mg capsule (product No. IC4541A) that is commercially available by Fujisawa was prepared.

### EXAMPLE 1

### Preparation of the Solid Dispersion of Tacrolimus with the Surfactant its HLB Value is About 7

Tacrolimus(1 g) was dissolved in the mixture of ethanol(10 ml) and dichloromethane(5 ml). To thus obtained solution, the sucrose fatty acid ester(HLB=7, 3 g) was dispersed as the drug carrier. The solution was evaporated under reduced pressure using a vacuum dryer. After drying, the residual product was pulverized.

### EXAMPLE 2

### Preparation of the Solid Dispersion of Tacrolimus with the Surfactant its HLB Value is About 9

Tacrolimus(1 g) was dissolved in the mixture of ethanol(10 ml) and dichloromethane(5 ml). To thus obtained solution, the sucrose fatty acid ester(HLB=9, 3 g) was dispersed as the drug carrier. The solution was evaporated under reduced pressure using a vacuum dryer. After drying, the residual product was pulverized.

### EXAMPLE 3

### Preparation of the Solid Dispersion of Tacrolimus with the Surfactant its HLB Value is About 11

Tacrolimus(1 g) was dissolved in the mixture of ethanol(10 ml) and dichloromethane(5 ml). To thus obtained solution, the sucrose fatty acid ester(HLB=11, 3 g) was dispersed as the drug carrier. The solution was evaporated under reduced pressure using a vacuum dryer. After drying, the residual product was pulverized.

### EXAMPLE 4

### Preparation of the Solid Dispersion of Tacrolimus with the Surfactant its HLB Value is About 15

Tacrolimus(1 g) was dissolved in the mixture of ethanol(10 ml) and dichloromethane(5 ml). To thus obtained solution, the sucrose fatty acid ester(HLB=15, 3 g) was dispersed as the drug carrier. The solution was evaporated under reduced pressure using a vacuum dryer. After drying, the residual product was pulverized.

### EXAMPLE 5

### Preparation of the Solid Dispersion of Tacrolimus with the Surfactant its HLB Value is About 16

Tacrolimus(1 g) was dissolved in the mixture of ethanol(10 ml) and dichloromethane(5 ml). To thus obtained solution, the sucrose fatty acid ester (HLB=16, 3 g) was dispersed as the drug carrier. The solution was evaporated under reduced pressure using a vacuum dryer. After drying, the residual product was pulverized.

### EXAMPLE 6

### Preparation of the Solid Dispersion of Tacrolimus with Sodium Lauryl Sulfate

Tacrolimus(1 g) was dissolved in the mixture of ethanol(10 ml) and dichloromethane(5 ml). To thus obtained solution, sodium lauryl sulfate(3 g) was dispersed as the drug carrier. The solution was evaporated under reduced pressure using a vacuum dryer. After drying, the residual product was pulverized.

### EXAMPLE 7

### Preparation of the Solid Dispersion of Tacrolimus with Poloxamer

Tacrolimus(1 g) was dissolved in the mixture of ethanol(10 ml) and dichloromethane(5 ml). To thus obtained solution, the poloxamer 188(3 g) was dispersed as the drug carrier. The solution was evaporated under reduced pressure using a vacuum dryer. After drying, the residual product was pulverized.

### EXAMPLE 8

### Preparation of the Solid Dispersion of Tacrolimus

Tacrolimus(1 g) was dissolved in the mixture of ethanol(10 ml) and dichloromethane(5 ml). To thus obtained solution, the sucrose fatty acid ester(HLB=9, 3 g) was dispersed as the drug carrier, and then was added croscarmellose sodium(7 g), additionally. The solution was evaporated under reduced pressure using a vacuum dryer. After drying, the residual product was pulverized.

### EXAMPLE 9

### Preparation of the Solid Dispersion of Tacrolimus

Tacrolimus(1 g) was dissolved in the mixture of ethanol(10 ml) and dichloromethane(5 ml). To thus obtained solution, sodium lauryl sulfate(3 g) was dispersed as the drug carrier, and then was added croscarmellose sodium(7 g), additionally. The solution was evaporated under reduced pressure using a vacuum dryer. After drying, the residual product was pulverized.

### EXAMPLE 10

### Preparation of the Solid Dispersion of Tacrolimus

Tacrolimus(1 g) was dissolved in the mixture of ethanol(10 ml) and dichloromethane(5 ml). To thus obtained solution, the poloxamer 188(3 g) was dispersed as the drug carrier, and then was added croscarmellose sodium(7 g), additionally. The solution was evaporated under reduced pressure using a vacuum dryer. After drying, the residual product was pulverized.

### EXAMPLE 11

### Preparation of the Solid Dispersion of Tacrolimus

Tacrolimus(1 g) was dissolved in the mixture of ethanol(10 ml) and dichloromethane(5 ml). To thus obtained solution, the sucrose fatty acid ester(HLB=9, 3 g) and sodium lauryl sulfate(3 g) were dispersed as the drug carrier. The solution was evaporated under reduced pressure using a vacuum dryer. After drying, the residual product was pulverized.

### EXAMPLE 12

### Preparation of the Solid Dispersion of Tacrolimus

Tacrolimus(1 g) was dissolved in the mixture of ethanol(10 ml) and dichloromethane(5 ml). To thus obtained solution, the sucrose fatty acid ester(HLB=9, 3 g) and the poloxamer 188(3 g) were dispersed as the drug carrier. The solution was evaporated under reduced pressure using a vacuum dryer. After drying, the residual product was pulverized.

### EXAMPLE 13

### Preparation of the Solid Dispersion of Tacrolimus

Tacrolimus(1 g) was dissolved in the mixture of ethanol(10 ml) and dichloromethane(5 ml). To thus obtained solution, sodium lauryl sulfate(3 g) and the poloxamer 188(3 g) were dispersed as the drug carrier. The solution was evaporated under reduced pressure using a vacuum dryer. After drying, the residual product was pulverized.

### EXAMPLE 14

### Preparation of the Solid Dispersion of Tacrolimus

Tacrolimus(1 g) was dissolved in the mixture of ethanol(10 ml) and dichloromethane(5 ml). To thus obtained solution, the sucrose fatty acid ester(HLB=9, 3 g) and sodium lauryl sulfate(3 g) were dispersed as the drug carrier, and then was added croscarmellose sodium(7 g), additionally. The solution was evaporated under reduced pressure using a vacuum dryer. After drying, the residual product was pulverized.

### EXAMPLE 15

### Preparation of the Solid Dispersion of Tacrolimus

Tacrolimus(1 g) was dissolved in the mixture of ethanol(10 ml) and dichloromethane(5 ml). To thus obtained solution, the sucrose fatty acid ester(HLB=9, 3 g) and the poloxamer 188(3 g) were dispersed as the drug carrier, and then was added croscarmellose sodium(7 g), additionally. The solution was evaporated under reduced pressure using a vacuum dryer. After drying, the residual product was pulverized.

### EXAMPLE 16

### Preparation of the Solid Dispersion of Tacrolimus

Tacrolimus(1 g) was dissolved in the mixture of ethanol(10 ml) and dichloromethane(5 ml). To thus obtained solution, sodium lauryl sulfate(3 g) and the poloxamer 188(3 g) were dispersed as the drug carrier, and then was added croscarmellose sodium(7 g), additionally. The solution was evaporated under reduced pressure using a vacuum dryer. After drying, the residual product was pulverized.

### EXAMPLE 17

### Preparation of the Solid Dispersion of Tacrolimus

Tacrolimus(30 g) was dissolved in the mixture of ethanol(100 ml) and dichloromethane(50 ml). To thus obtained solution, the sucrose fatty acid ester(HLB=9, 90 g) was dispersed as the drug carrier. The solution was sprayed on the talc(300 g) that was fluidified in fluid bed granulator, and then dried.

### EXAMPLE 18

### Preparation of the Solid Dispersion of Tacrolimus

Tacrolimus(30 g) was dissolved in the mixture of ethanol(100 ml) and dichloromethane(50 ml). To thus obtained solution, the sucrose fatty acid ester(HLB=9, 90 g) was dispersed as the drug carrier. The solution was sprayed on anhydrous dibasic calcium phosphate(300 g) that was fluidified in fluid bed granulator, and then dried.

### EXAMPLE 19

### Preparation of the Solid Dispersion of Tacrolimus

Tacrolimus(30 g) was dissolved in the mixture of ethanol(100 ml) and dichloromethane(50 ml). To thus obtained solution, the sucrose fatty acid ester(HLB=9, 90 g) was dispersed as the drug carrier. The solution was sprayed on lactose(300 g) that was fluidified in fluid bed granulator, and then dried.

### EXAMPLE 20

### Preparation of the Solid Dispersion of Tacrolimus

Tacrolimus(30 g) was dissolved in the mixture of ethanol(100 ml) and dichloromethane(50 ml). T6 thus obtained solution, sodium lauryl sulfate(90 g) was dispersed as the drug carrier. The solution was sprayed on talc(300 g) that was fluidified in fluid bed granulator, and then dried.

### EXAMPLE 21

### Preparation of the Solid Dispersion of Tacrolimus

Tacrolimus(30 g) was dissolved in the mixture of ethanol(100 ml) and dichloromethane(50 ml). To thus obtained solution, sodium lauryl sulfate(90 g) was dispersed as the drug carrier. The solution was sprayed on anhydrous dibasic calcium phosphate(300 g) that was fluidified in fluid bed granulator, and then dried.

### EXAMPLE 22

### Preparation of the Solid Dispersion of Tacrolimus

Tacrolimus(30 g) was dissolved in the mixture of ethanol(100 ml) and dichloromethane(50 ml). To thus obtained solution, sodium lauryl sulfate(90 g) was dispersed as the drug carrier. The solution was sprayed on lactose(300 g) that was fluidified in fluid bed granulator, and then dried.

### EXAMPLE 23

### Preparation of the Solid Dispersion of Tacrolimus

Tacrolimus(30 g) was dissolved in the mixture of ethanol(100 ml) and dichloromethane(50 ml). To thus obtained solution, the sucrose fatty acid ester(HLB=9, 90 g) was dispersed as the drug carrier. The solution was sprayed on talc(300 g) that was fluidified in fluid bed granulator, and then dried.

### EXAMPLE 24

### Preparation of the Solid Dispersion of Tacrolimus

Tacrolimus(30 g) was dissolved in the mixture of ethanol(100 ml) and dichloromethane(50 ml). To thus obtained solution, sodium lauryl sulfate(90 g) and the sucrose fatty acid ester(HLB=9, 90 g) were dispersed as the drug carrier. The solution was sprayed on anhydrous dibasic calcium phosphate(300 g) that was fluidified in fluid bed granulator, and then dried.

### EXAMPLE 25

### Preparation of the Solid Dispersion of Tacrolimus

Tacrolimus(30 g) was dissolved in the mixture of ethanol(100 ml) and dichloromethane(50 ml). To thus obtained solution, sodium lauryl sulfate(90 g) and the sucrose fatty acid ester(HLB=9, 90 g) were dispersed as the drug carrier. The solution was sprayed on lactose(300 g) that was fluidified in fluid bed granulator, and then dried.

### EXAMPLE 26

### Preparation of the Solid Dispersion of Tacrolimus

Tacrolimus(30 g) was dissolved in the mixture of ethanol(100 ml) and dichloromethane(50 ml). To thus obtained solution, sodium lauryl sulfate(90 g) and the sucrose fatty acid ester(HLB=9, 90 g) were dispersed as the drug carrier, and then was added croscarmellose sodium(210 g), additionally. The solution was sprayed on anhydrous dibasic calcium phosphate(300 g) that was fluidified in fluid bed granulator, and then dried.

### EXAMPLE 27

### Preparation of the Solid Dispersion of Tacrolimus

Tacrolimus(30 g) was dissolved in the mixture of ethanol(100 ml) and dichloromethane(50 ml). To thus obtained solution, the sucrose fatty acid ester(HLB=9, 90 g) was dispersed as the drug carrier, and then was added croscarmellose sodium(210 g), additionally. The solid dispersion was prepared by spray drying of the solution.

### EXAMPLE 28

### Preparation of the Solid Dispersion of Tacrolimus

Tacrolimus(30 g) was dissolved in the mixture of ethanol(100 ml) and dichloromethane(50 ml). To thus obtained solution, sodium lauryl sulfate(90 g) was dispersed as the drug carrier, and then was added croscarmellose sodium(210 g), additionally. The solid dispersion was prepared by spray drying of the solution.

### EXAMPLE 29

### Preparation of the Solid Dispersion of Tacrolimus

Tacrolimus(30 g) was dissolved in the mixture of ethanol(100 ml) and dichloromethane(50 ml). To thus obtained solution, the sodium lauryl sulfate(90 g) and the sucrose fatty acid ester(HLB=9, 90 g) were dispersed as the drug carrier, and then was added croscarmellose sodium(210 g), additionally. The solid dispersion was prepared by spray drying of the solution.

### PREPARATION EXAMPLE 1

### Preparation of the Tacrolimus Capsule

Each solid dispersion include tacrolimus 1 mg(prepared in Comparative examples 1 and 2, and examples from 1 to 29) was mixed with anhydrous lactose, croscarmellose sodium, and magnesium stearate. The mixtures were filled into a gelatin capsule, respectively.

### PREPARATION EXAMPLE 2

### Preparation of the Tacrolimus Tablet

Each solid dispersion include tacrolimus 1 mg(prepared in Comparative examples 1 and 2, and examples from 1 to 29) was mixed with anhydrous lactose, microcrystalline cellulose, croscarmellose sodium, and magnesium stearate. The mixtures were formulated into tablet, respectively.

### EXPERIMENTAL EXAMPLE 1

### Dissolution Test

The Dissolution tests was performed in accordance with method 2(Paddle method) of the Korean Pharmacopoeia(KP). As the test solution, 900 mL of 0.005%(w/v) hydroxypropylcellulose solution was used. The paddle speed was set to 50 rpm. The prograf 1 mg capsules in Comparative example 3 and the capsules and the tablets prepared in Preparation examples 1 and 2 were added to the test solutions and after 5, 10, 15, 30 and 60 minutes, the test solutions were taken as samples. They were analyzed by high-performance liquid chromatography. The results were represented in Table 1 and 2.

TABLE 1
Dissolution rate(%) of the tacrolimus capsules prepared in
Preparation example 1

| | dissolution rate(%) | | | | |
|---|---|---|---|---|---|
| | 5 min | 10 min | 15 min | 30 min | 60 min |
| comparative example 1 | 4.4 | 9.8 | 15.6 | 28.1 | 34.3 |
| comparative example 2 | 12.4 | 19.5 | 26.5 | 39.7 | 48.9 |
| comparative example 3 | 4.6 | 12.4 | 21.5 | 38.9 | 65.8 |
| example 1 | 53.4 | 59.6 | 64.5 | 70.3 | 72.9 |
| example 2 | 63.5 | 71.3 | 73.8 | 75.1 | 77.9 |
| example 3 | 58.1 | 61.3 | 65.7 | 69.5 | 71.7 |
| example 4 | 57.9 | 61.4 | 65.1 | 69.9 | 71.5 |
| example 5 | 58.1 | 60.3 | 64.6 | 70.6 | 72.6 |
| example 6 | 67.3 | 71.7 | 74.2 | 76.4 | 77.8 |
| example 7 | 60.4 | 64.6 | 65.5 | 68.2 | 69.1 |
| example 8 | 68.5 | 83.4 | 83.6 | 84.4 | 84.2 |
| example 9 | 71.1 | 84.2 | 84.5 | 85.2 | 85.8 |
| example 10 | 63.4 | 65.6 | 67.8 | 68.3 | 69.4 |
| example 11 | 67.5 | 72.3 | 74.3 | 76.7 | 78.8 |
| example 12 | 63.4 | 71.3 | 73.5 | 75.2 | 77.7 |
| example 13 | 67.2 | 71.6 | 74.4 | 76.1 | 77.9 |
| example 14 | 75.3 | 85.5 | 87.6 | 88.9 | 90.1 |
| example 15 | 63.5 | 72.5 | 79.8 | 84.4 | 84.5 |
| example 16 | 64.8 | 75.3 | 79.2 | 85.4 | 85.4 |
| example 17 | 63.2 | 71.3 | 73.6 | 75.1 | 78.8 |
| example 18 | 62.8 | 72.2 | 73.1 | 75.3 | 79.3 |
| example 19 | 63.2 | 72.1 | 73.7 | 75.2 | 77.6 |
| example 20 | 67.5 | 71.7 | 74.5 | 76.5 | 78.8 |
| example 21 | 64.4 | 71.4 | 74.2 | 75.6 | 79.3 |
| example 22 | 64.9 | 73.2 | 73.5 | 75.2 | 77.6 |
| example 23 | 67.5 | 72.4 | 74.5 | 76.7 | 78.8 |
| example 24 | 68.3 | 73.1 | 74.3 | 77.7 | 79.5 |
| example 25 | 68.2 | 72.3 | 74.9 | 75.3 | 77.1 |
| example 26 | 75.5 | 85.7 | 87.3 | 88.7 | 90.1 |
| example 27 | 68.2 | 83.1 | 83.4 | 84.3 | 84.4 |
| example 28 | 71.0 | 84.5 | 84.8 | 85.7 | 85.7 |
| example 29 | 75.7 | 85.6 | 87.1 | 88.8 | 90.2 |

TABLE 2
Dissolution rate(%) of the tacrolimus tablets prepared in
Preparation example 2

| | dissolution rate(%) | | | | |
|---|---|---|---|---|---|
| | 5 min | 10 min | 15 min | 30 min | 60 min |
| comparative example 1 | 2.4 | 7.8 | 14.6 | 27.1 | 34.1 |
| comparative example 2 | 5.4 | 10.5 | 21.5 | 35.7 | 46.9 |
| comparative example 3 | 4.6 | 12.4 | 21.5 | 38.9 | 65.8 |
| example 1 | 50.1 | 55.6 | 62.5 | 68.3 | 72.5 |
| example 2 | 59.3 | 67.4 | 71.8 | 73.1 | 76.4 |
| example 3 | 55.1 | 58.3 | 62.7 | 67.5 | 70.7 |
| example 4 | 53.8 | 57.2 | 62.1 | 68.9 | 70.5 |
| example 5 | 53.1 | 55.3 | 61.2 | 69.6 | 71.9 |
| example 6 | 64.3 | 68.7 | 72.1 | 75.4 | 77.1 |
| example 7 | 57.2 | 61.5 | 63.7 | 66.2 | 68.6 |
| example 8 | 65.5 | 81.4 | 81.6 | 83.1 | 83.7 |
| example 9 | 68.1 | 81.2 | 81.5 | 84.2 | 85.2 |
| example 10 | 60.3 | 62.6 | 65.1 | 67.3 | 69.0 |
| example 11 | 64.5 | 68.2 | 72.3 | 74.9 | 77.9 |
| example 12 | 60.5 | 68.3 | 71.3 | 74.1 | 77.5 |
| example 13 | 64.1 | 68.5 | 72.0 | 75.4 | 77.1 |
| example 14 | 72.1 | 82.4 | 85.5 | 87.9 | 89.9 |
| example 15 | 60.1 | 69.3 | 77.8 | 83.1 | 84.4 |
| example 16 | 61.5 | 72.7 | 77.1 | 84.6 | 85.9 |
| example 17 | 60.5 | 67.9 | 70.7 | 74.5 | 78.9 |
| example 18 | 59.2 | 68.6 | 71.2 | 74.3 | 79.1 |
| example 19 | 60.2 | 68.7 | 70.7 | 74.0 | 77.9 |
| example 20 | 64.2 | 68.2 | 70.4 | 74.0 | 78.5 |
| example 21 | 61.5 | 68.4 | 71.2 | 74.6 | 78.9 |
| example 22 | 61.4 | 70.8 | 72.5 | 74.8 | 77.7 |
| example 23 | 64.3 | 69.4 | 73.5 | 75.2 | 78.5 |
| example 24 | 64.3 | 69.1 | 72.9 | 75.7 | 79.1 |
| example 25 | 64.2 | 68.5 | 72.8 | 73.6 | 77.7 |
| example 26 | 72.5 | 79.7 | 84.3 | 87.7 | 91.1 |
| example 27 | 64.2 | 79.2 | 81.3 | 83.1 | 84.0 |
| example 28 | 68.0 | 79.5 | 81.8 | 84.7 | 85.5 |
| example 29 | 72.3 | 80.6 | 85.1 | 87.7 | 89.2 |

As a result, the maximum dissolution rates (%) of the capsules and the tablets prepared in the Preparation examples 1 and 2 were greater than or equal to about 65%.

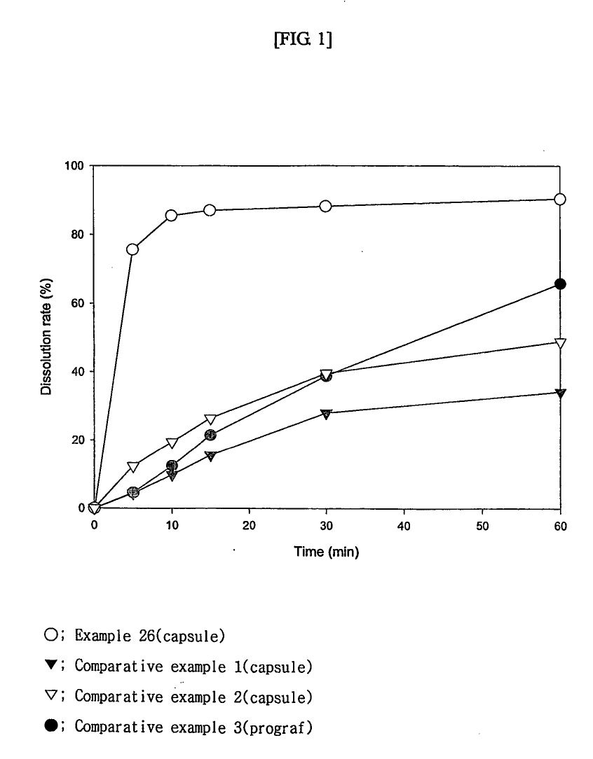

The dissolution rate of the present invention is higher than that of the commercially available dosage form prepared in Comparative example 3 (see FIG. 1).

So, the tacrolimus dosage form prepared by using the above-prepared solid dispersion has the rapid drug release, and the bioavailability and the oral absorbability of the dosage form may be increased due to the excellent dissolution rate of tacrolimus.

But the solid dispersion prepared in Comparative examples 1 and 2 did not show the rapid drug release. Therefore, the surfactant having a property of the HLB value less than 7 is not preferred for the preparation of the solid dispersion in the present invention.

1. A solid dispersion comprising tacrolimus and solid surfactant having a property of hydrophile lipophile balance (HLB) value higher than or equal to about 7.

2. The solid dispersion according to claim 1, wherein the surfactant is at least one selected from the group consisting of sodium lauryl sulfate (HLB=40), poloxamers (HLB≧7), and sucrose fatty acid esters (18≧HLB≧7).

3. The solid dispersion according to claim 1, the tacrolimus and the solid surfactant are mixed by weight in a ratio of about 1:0.1 to about 1:100.

4. The solid dispersion according to any one of claim 1 through claim 3, comprising additives, without a function of a carrier, of more than one selected from the group consisting of pharmaceutically acceptable excipients, disintegrators, coloring agents, flavouring agents, sweetening agents and lubricants.

5. A method of processing a solid dispersion comprising;
dissolving or dispersing tacrolimus and solid surfactant (HLB≧7) in solvent that is at least one selected from the group consisting of ethanol, isopropyl alcohol, dichloromethane and chloroform to produce a solution; and,
drying the solution.

6. The method of claim 5, further comprising;
adding additives, without a function of a carrier, of at least one selected from the group consisting of pharmaceutically acceptable excipients, disintegrators, coloring agents, flavouring agents, sweeting agents and lubricants to the solution.

7. A method of processing a solid dispersion, comprising;
dissolving or dispersing tacrolimus and solid surfactant (HLB≧7) in solvent that is at least one selected from the group consisting of ethanol, isopropyl alcohol, dichloromethane and chloroform to produce a solution; and
spraying the solution on additives, without a function of the carrier, of at least one selected form the group consisting of pharmaceutically acceptable excipients, disintegrators, coloring agents, flavouring agents, sweetening agents and lubricants for producing a granule.
